(19) United States
(12) Patent Application Publication
(10) Pub. No.: US 2024/0358292 A1
(43) Pub. Date: Oct. 31, 2024

(54) POLARIZED PHOTOPLETHYSMOGRAPHY (PPG) WITH IMPROVED PERFUSION SIGNAL FOR ADVANCED BIOSENSING

(71) Applicant: Samsung Electronics Co., Ltd., Suwon-si (KR)

(72) Inventors: Shailabh Kumar, Pasadena, CA (US); Radwanul Hasan Siddique, Monrovia, CA (US)

(21) Appl. No.: 18/767,471

(22) Filed: Jul. 9, 2024

Related U.S. Application Data
(63) Continuation-in-part of application No. 18/103,855, filed on Jan. 31, 2023.
(60) Provisional application No. 63/517,014, filed on Aug. 1, 2023.
(60) Provisional application No. 63/610,151, filed on Dec. 14, 2023.
(60) Provisional application No. 63/400,213, filed on Aug. 23, 2022.
(60) Provisional application No. 63/421,710, filed on Nov. 2, 2022.

(51) Int. Cl. A61B 5/1455; A61B 5/00; A61B 5/0205; A61B 5/026

(52) U.S. Cl. CPC A61B 5/14552; A61B 5/0205; A61B 5/7264; A61B 5/7278; A61B 5/0261; A61B 2562/0238; A61B 2562/0242; A61B 2562/046

(57) ABSTRACT

A biometric measurement device including a photodiode array having a number of photodiodes and at least one polarization filter covering at least one of the photodiodes. The biometric measurement device also includes a light-emitting diode array having a number of light-emitting diodes proximate to the photodiode array, a non-volatile memory device, and a processor in electronic communication with the non-volatile memory device. The photodiode array is configured to receive a reflected waveform from light emitted from the light-emitting diode array, and the processor is configured to determine at least one physiological parameter based on a pulsatile (AC) component and a static (DC) component of the reflected waveform. The light-emitting diodes may emit polarized light.

### CROSS-REFERENCE TO RELATED APPLICATION(S)

The present application is a continuation-in-part of U.S. application Ser. No. 18/103,855, filed Jan. 31, 2023, which claims priority to and the benefit of U.S. Provisional Patent Application No. 63/400,213, filed Aug. 23, 2022, and U.S. Provisional Patent Application No. 63/421,710, filed Nov. 2, 2022. The present application also claims priority to and the benefit of U.S. Provisional Application No. 63/610,151, filed Dec. 14, 2023. The entire content of each of these applications is incorporated herein by reference.

### BACKGROUND

### 1. Field

The present disclosure relates to devices and methods for performing photoplethysmography.

### 2. Description of the Related Art

Photoplethysmography (PPG) is a non-contact, non-invasive optical imaging technique which uses intensity and color-based information of transmitted or reflected light to measure various physiological parameters, such as blood pressure and cardiovascular markers. However, related art devices and methods of performing PPG have a relatively high signal-to-noise ratio and signal artifacts that hinder biosignal analysis and the accurate measurement of certain health conditions.

The above information disclosed in this Background section is only for enhancement of understanding of the background of the invention and therefore it may contain information that does not constitute prior art.

### SUMMARY

The present disclosure relates to various embodiments of a biometric measurement device. In one embodiment, the biometric measurement device includes a photodiode array having photodiodes and at least one polarization filter covering at least one of the photodiodes. The biometric measurement device also includes a light-emitting diode array having light-emitting diodes proximate to the photodiode array, a non-volatile memory device, and a processor in electronic communication with the non-volatile memory device. The photodiode array is configured to receive a reflected waveform from light emitted from the light-emitting diode array, and the processor is configured to determine at least one physiological parameter based on a pulsatile (AC) component and a static (DC) component of the reflected waveform.

The present disclosure also relates to various embodiments of a method of performing photoplethysmography. In one embodiment, the method includes emitting light from at least one light-emitting diode of a number of light-emitting diodes arranged in a light-emitting diode array; receiving a polarized waveform, with at least one photodiode of a number of photodiodes arranged in a photodiode array, from reflected light of the light emitted by the light-emitting diode; and determining at least one physiological parameter based on a pulsatile (AC) component and a static (DC) component of the polarized waveform.

This summary is provided to introduce a selection of concepts that are further described below in the detailed description. This summary is not intended to identify key or essential features of the claimed subject matter, nor is it intended to be used in limiting the scope of the claimed subject matter. One or more of the described features may be combined with one or more other described features to provide a workable device.

### BRIEF DESCRIPTION OF THE DRAWINGS

The features and advantages of embodiments of the present disclosure will be better understood by reference to the following detailed description when considered in conjunction with the drawings. The drawings are not necessarily drawn to scale.

FIGS. 1A-1D depict a watch, a card, a ring, and a finger pulse oximeter, respectively, configured to perform photoplethysmography (PPG) according to embodiments of the present disclosure;

FIG. 1E is a schematic view depicting light from an LED reflecting off a vessel below subcutaneous tissue underneath the user's skin, and being captured by a photodiode (PD) according to one embodiment of the present disclosure;

FIGS. 2A-2B depict two different configurations (out of other possible configurations) of light-emitting diodes and photodiodes according to embodiments of the present disclosure;

FIGS. 3A-3B are schematic diagrams depicting operation of the wearable biometric measurement device according to one embodiment of the present disclosure in which polarized light is emitted from an LED, reflected off a user's blood vessel, and received at a photodiode with a single polarizer pixel and/or an array of polarizer pixels;

FIG. 3C is a comparative example of a related art device in which non-polarized light is emitted from an LED, reflected off a user's blood vessel, and received at a photodiode without a polarizer;

FIG. 4A is a photograph of the specular reflection off a device/tube mimicking a user's blood vessel when the device/tube mimicking the user's blood vessel is irradiated with polarized light;

FIG. 4B is a photograph of the specular reflection off a device/tube mimicking a user's blood vessel when the device/tube mimicking the user's blood vessel is irradiated with non-polarized light;

FIG. 5 is a graph depicting the waveform reflected from a user's blood vessel, which includes a pulsatile (AC) component and a static (DC) component;

FIGS. 6A-6B are graphs depicting the perfusion index of the reflected waveform when the blood vessel is irradiated with polarized light and non-polarized light, respectively;

FIG. 7 is a graph depicting the perfusion index (PI) ratio as a function of LED power according to one embodiment of the present disclosure;

FIG. 8A is a graph depicting the perfusion index (%) of the photodiode with and without a polarization filter for each of six human subjects having different skin tones;

FIG. 8B is a graph depicting the signal-to-noise ratio (SNR) of the photodiode with and without a polarization filter for each of the six human subjects having different skin tones; and

FIG. 9 depicts an an LED and a pair of photodiodes on opposite sides of the LED arranged in a linear array according to one embodiment of the present disclosure;

FIG. 10 is a flowchart illustrating tasks of a method of performing photoplethysmography according to one embodiment of the present disclosure; and

FIG. 11 is a flowchart illustrating an artificial intelligence-based method of performing photoplethysmography according to one embodiment of the present disclosure.

### DETAILED DESCRIPTION

The present disclosure relates to various devices and methods of performing photoplethysmography (PPG) to measure one or more physiological parameters of a user, such as continuous blood pressure, cardiovascular markers, skin health, metabolism, and/or nutrition. In one or more embodiments, the devices and methods of the present disclosure irradiate a portion of a user's blood vessel with light (e.g., polarized light) emitted from a light source and measure the reflected light with a light sensor having a polarization filter. The use of polarized light is configured to reduce specular reflection and thereby reduce signal artifacts and improve the signal-to-noise ratio. The devices and methods of the present disclosure also utilize a signal collection algorithm that calculates the perfusion index (PI), which is the ratio of the pulsatile (AC) component of the signal waveform (due to pumping action of the heart and resultant change in blood volume in vessels) and the static (DC) component of the signal waveform (from the residual blood in vessels as well as reflection from skin, tissue, and bones), and utilizes the PI to calculate one or more physiological parameters of the user.

Hereinafter, example embodiments will be described in more detail with reference to the accompanying drawings, in which like reference numbers refer to like elements throughout. The present invention, however, may be embodied in various different forms, and should not be construed as being limited to only the illustrated embodiments herein. Rather, these embodiments are provided as examples so that this disclosure will be thorough and complete, and will fully convey the aspects and features of the present invention to those skilled in the art. Accordingly, processes, elements, and techniques that are not necessary to those having ordinary skill in the art for a complete understanding of the aspects and features of the present invention may not be described. Unless otherwise noted, like reference numerals denote like elements throughout the attached drawings and the written description, and thus, descriptions thereof may not be repeated.

In the drawings, the relative sizes of elements, layers, and regions may be exaggerated and/or simplified for clarity. Spatially relative terms, such as “beneath,” “below,” “lower,” “under,” “above,” “upper,” and the like, may be used herein for ease of explanation to describe one element or feature's relationship to another element(s) or feature(s) as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present invention.

The terminology used herein is for the purpose of describing particular embodiments and is not intended to be limiting of the present invention. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

As used herein, the term “substantially,” “about,” and similar terms are used as terms of approximation and not as terms of degree, and are intended to account for the inherent variations in measured or calculated values that would be recognized by those of ordinary skill in the art. Further, the use of “may” when describing embodiments of the present invention refers to “one or more embodiments of the present invention.” As used herein, the terms “use,” “using,” and “used” may be considered synonymous with the terms “utilize,” “utilizing,” and “utilized,” respectively. Also, the term “exemplary” is intended to refer to an example or illustration.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

For the purposes of this disclosure, expressions such as “at least one of,” when preceding a list of elements, modify the entire list of elements and do not modify the individual elements of the list. For example, “at least one of X, Y, and Z,” “at least one of X, Y, or Z,” and “at least one selected from the group consisting of X, Y, and Z” may be construed as X only, Y only, Z only, any combination of two or more of X, Y, and Z, such as, for instance, XYZ, XYY, YZ, and ZZ, or any variation thereof. Similarly, the expression such as “at least one of A and B” may include A, B, or A and B. As used herein, “or” generally means “and/or,” and the term “and/or” includes any and all combinations of one or more of the associated listed items. For example, the expression such as “A and/or B” may include A, B, or A and B.

In this disclosure, a light (e.g., electromagnetic spectral emission) detection position can be at different locations (e.g., spatially varying, tilted, etc.). In addition, a light source can have collimator elements, diffractive optical elements, etc.

Furthermore, multiple methods of light detection and analysis are described herein. For example, different types of polarized and non-polarized light may be emitted and analyzed to provide biosignals for reducing an SNR and improve detection of bodily parameters.

According to an embodiment, the present disclosure provides a method including the steps of emitting light (e.g., polarized light), collecting the light, collecting attributes from the collected light (e.g., attributes regarding a lighting intensity at a certain polarization states and a wavelength in a range, and for multiple pixels (multispectral)), calculating Stokes parameters, calculating signals based on the Stokes parameters, repeating some or all of the abovementioned steps, and analyzing the data.

The emitted light can be one or more of collimated light, diffused light, polarized light, multispectral light, pulsed light, continuous light, in the visible or infrared spectrum. Collecting the light may be performed on at least one pixel.

The attributes from the collected light can be one or more of a spectrum, a polarization state, a light intensity, and a depth (e.g., a time of flight).

At least one pixel can detect both a wavelength range (e.g., 0.5 nanometers (nm) to 400 nm) and one polarization state, and can have hardware filters (nanostructures or others) for allowing the wavelength range and polarization state to pass through.

Calculating the Stokes parameters may be done for at least one pixel using intensity data from the pixel that had a polarization state and wavelength range filter.

The signals calculated may include at least one of a depolarization state (e.g., a degree of polarization changed from an initial to a final state), retardance (e.g., an angle of polarization), diattenuation (e.g., a reduction in intensity), spectral changes, and a depth estimation.

The method can be used for gathering information related to biosignal detection. This information may include, and is not limited to, a heart rate (HR), a respiratory rate, hypertension signatures, a red blood cell concentration, a blood saturation level, continuous blood pressure, a pulse rate, a pulse pressure, cardiovascular conditions, stroke volume, a cardiac output, a one lead electrocardiogram (ECG), a systematic vascular resistance, a cardiac index, a mean arterial pressure, antioxidants, melanoma, triglyceride, cholesterol, and/or beta carotene.

Accordingly, the present disclosure provides methods and systems for a polarization controlled light source and multispectral full-Stokes (linear and circular) polarization PPG sensors.

The present disclosure may modulate the phase and polarization of the light simultaneously to improve the signal-to-background ratio (SBR) and angle-dependent properties.

Polarization and multispectral information may be obtained for detecting polarization and spectral sensitive physiological parameters and molecular information of antioxidants, melanoma, triglyceride, and cholesterol.

The present disclosure advantageously provides an on-chip design with high efficiency with detection of all polarization states, focusing light using the filters with reduced angle-dependency and a high field of view (FOV). In addition, the present disclosure may reduce signal to background noise and detect polarization sensitive targets/molecules.

Accordingly, methods and systems for biosensing using a PPG sensor system, analysis, and feedback ecosystem are provided. A polarization controlled light source (a light emitting device (LED), a laser diode, or a vertical-cavity surface-emitting laser (VCSEL)) with passive or active polarization filters (nanostructured and/or liquid crystals) may be provided. Additionally, a compact PPG sensor system may be provided that utilizes complete on-chip polarization, and multispectral sensors (PD, avalanche photodiode (APD), and single-photon avalanche photodiode (SPAD)). Additionally, a suite of detection and analysis algorithms that utilize the multiple sensors may be provided. Further, an integrated feedback loop system that includes measuring the body's physiological parameter response to polarized light is provided.

A method and system of using polarization-controlled light source and multispectral full-Stokes polarization PPG sensors may be provided. The system may modulate the phase and polarization of the light simultaneously to improve SBR and angle-dependent field of view (FOV) properties.

Polarization and multispectral information for detecting polarization and spectral sensitive physiological parameters and molecular information of antioxidants, melanoma, triglyceride, and cholesterol may also be provided.

Accordingly, a highly efficient on-chip design, for all detectable polarization states focusing light using the filters, reduced angle-dependency and high FOV, reduced signal to background noise, and detecting polarization sensitive targets/molecules may be provided.

A PPG sensor system uses a non-contact non-invasive optical imaging technique which uses intensity and color-based information of transmitted or reflected light to measure physiological parameters. For example, a PPG sensor system may record a reflected/transmitted time-varying signal. Temporal analysis of spectrum and intensity information of the transmitted or reflected light may include important health-related information. In addition, PPG may be used for blood pulsation and HR measurements, utilized to obtain cardiovascular and respiratory information.

In some embodiments, a light emitting diode (LED) may emit light towards the skin of the user through the subcutaneous tissue, and may be reflected off of the blood vessel. The reflected light may be received by a PD and include a particular pulse wave or other bioinformation. Characteristics of the pulse wave may reveal several different types of physiological information (or patterns) about the user (e.g., blood pulsation and HR measurements, cardiovascular and respiratory information, etc.).

Light reflected from the patient's skin and captured by the PPG sensor system may include three components:
Light that is a direct reflection from the skin surface. This type of light may have a reduced signal to noise ratio (SNR).
Light that is a reflection from superficial layer of tissues, scattering through the epidermis cuticles. This type of light may impede or interfere with important/relevant biosignals.
Backscattered light from deep layer Dermis tissues. This type of light may be weak to capture, and may include hard to classify signals among different molecules in the layer.

Optical measurements with PPG sensors of skin microcirculation (e.g., blood volume and flow) may provide continuous readout information of critical parameters including heartrate and oxygen saturation (O₂ saturation), and may indicate health conditions including hypertension, cardiovascular ailments, and anemia if, for example, a higher and improved SBR is made possible with deeper penetration of light through skin.

In addition, wearable PPG sensors may face various foundational challenges such as optical noise (e.g., scattering/reflection and no collimation); challenges arising based on the sensor location on the body (e.g., wrist versus ear versus arm); skin tone (e.g., some skin tones provide less signal absorption and less penetration); the crossover problem (e.g., artifacts cause by motion/activity); and low perfusion (e.g., issues related to obesity, diabetes, heart conditions, and arterial diseases each lowering blood perfusion).

Accordingly, measurements may be difficult and inaccurate due to interfering signals, such as direct reflection from the skin surface or tissues, backscattered light from deep dermal tissues, and/or motion-related artifacts.

A method and system for using a compact PPG sensor system that utilizes on-chip polarization characteristics and on-chip multispectral sensor characteristics may be provided. “On-chip” may mean that components that are described as “on-chip” are directly included as a part of the chip. That is, on-chip polarizing filters for both a light source and a light sensor may be used, which can be readily fabricated, scaled, and incorporated monolithically in wearable devices, thereby improving the accuracy of measurements by removing (or reducing) the effects of direct reflection and scattering, and removing (or reducing motion-artifact related noise).

Polarization sensors combined with signal processing can help isolate/reveal signal(s) from different biological components, such as blood, skin antioxidants, fat tissue, and more. These improvements can be used to predict ailments, such as hypertension, cardiovascular ailments, anemia and more.

The PPG sensor system may include a polarization controlled light source (e.g., an LED, laser diode or VCSELL) with passive or active polarization optics (e.g., nanostructures or liquid crystals). The polarization optics may be static (where a polarization state is predetermined by the filter or dynamic (where a polarization state can be changed by the filter). The method and system may simultaneously provide an emission using a polarization controlled light source and detect a multispectral full-Stokes polarization using PPG sensors. Accordingly, the method and system may simultaneously measure multiple polarization states emitted by an electromagnetic spectral emission source (e.g., the polarization controlled light source).

The method and system may use a detector to detect multiple states of polarization. A polarization state may refer to a polarization type or polarization angle of an electromagnetic spectral emission. For example, a polarization state may be linear polarization, circular polarization, or elliptical polarization.

The systems and methods of the present disclosure may detect an electromagnetic signal without polarization or with polarization.

In some embodiments, when an electromagnetic signal without polarization is detected, the amplitude of the detected signal varies significantly, and particularly when the source of the signal is moving.

In some embodiments, when an electromagnetic signal with polarization is detected, the amplitude of the detected signal is relatively consistent, even when the source of the signal is moving.

The improved detection efficiency may also be referred to as an improved SNR or an improved SBR.

Some embodiments may include an arrangement of four polarizing filters.

In some embodiments, the four polarizing filters respectively correspond to the four pixels. Each respective pixel includes a photodetector. The filter horizontally polarizes light passing through the filter. The filter vertically polarizes light. The filter diagonally polarizes light, and the filter circularly polarizes light. In an alternative embodiment, six polarizing filters and six pixels may be used. The additional two polarizing filters could be an anti-diagonally polarizing filter and a circularly polarizing filter that may polarize light in the opposite circular direction from the circularly polarizing filter.

The polarizing filter includes a wire grid and one or more phase-modulating nanostructures or metasurfaces. The wires of the wire grid may include a metal-insulator-metal (MIM) structure that suppresses reflection from cross-polarization. The nanostructures may be formed from a high dielectric index material, such as silicon (a Si, c Si, p-Si), silicon nitride (Si3N4), titanium dioxide (TiO2), Gallium nitride (GaN), Zinc oxide (ZnO), hafnium silicate, zirconium silicate, hafnium dioxide and zirconium dioxide. The nanostructures may also reduce the backscattering of the incident light, and may also help detect circular polarization.

The wire grid horizontally polarizes the light passing through the polarizing filter, and the nanostructures change, or modulate, the phase of the light that passes through the polarizing filter. The other polarizing filters also include a wire grid having a series of MIM structures and one or more nanostructures. The nanostructures of the circularly polarizing filter provide a 90-degree phase shift so that the circularly polarizing filter operates as a quarter wave plate. The nanostructures may modify wavelengths and be positioned with thin films to provide spacing for the nanostructures. The pixels may have additional antireflective thin film layers and microlens to improve the light collection.

The arrangement of the four polarizing filters and the four pixels may be used to generate the following six polarization states for incident light, shown in Equations (1)-(6), below.

$$\begin{matrix}{I}_{H} = {I}_{H} & Equation ( 1 ) \\ {I}_{V} = {I}_{V} & Equation ( 2 ) \\ {I}_{D} = {I}_{D} & Equation ( 3 ) \\ {I}_{A} = {I}_{H} + {I}_{V} - {I}_{D} & Equation ( 4 ) \\ {I}_{R} = {I}_{H} + {I}_{V} - {I}_{L} & Equation ( 5 ) \\ {I}_{L} = {I}_{L} & Equation ( 6 )\end{matrix}$$

in which I_{H} is the light intensity parameter of horizontally polarized light (H) measured at pixel, I_{V} is the light intensity parameter of the vertically polarized light (V) measured at pixel, I_{D} is the light intensity parameter of the diagonally polarized light (D) measured at pixel, I_{A} is the intensity parameter of the anti-diagonally polarized light, I_{R} is the intensity parameter of the right-hand circularly polarized light, and I_{L} is the light intensity parameter of the left-hand circularly polarized light (L) measured at pixel.

Using the intensity parameters of Equations (1)-(6), the Stokes parameters S₀, S₁, S₂ and S₃ may be calculated as follows, as shown below in Equations (7)-(14).

$$\begin{matrix}{S}_{0} = {I}_{H} + {I}_{V} & Equation ( 7 ) \\ {S}_{1} = {I}_{H} - {I}_{V} & Equation ( 8 ) \\ {S}_{2} = 2 * {I}_{D} - {S}_{0} & Equation ( 9 ) \\ {S}_{3} = 2 * {I}_{L} - S 0 & Equation ( 10 ) \\ DoLP = sqrt ( {S}_{1}^{2} + {S}_{2}^{2} ) / {S}_{0} & Equation ( 11 ) \\ AoLP = \text{.5} * tan - 1 * {S}_{2} / {S}_{1} & Equation ( 12 ) \\ DoCP = {S}_{3} / {S}_{0} & Equation ( 13 ) \\ AoCP = \text{.5} * {sin}^{- 1} * {S}_{3} / {S}_{1} & Equation ( 14 )\end{matrix}$$

in which DoLP is the degree of linear polarization, AoLP is the angle of linear polarization, DOCP is the degree of circular polarization, and AoCP is the angle of circular polarization.

In some embodiments, the polarizer may detect up to six polarization states. The polarizer includes four polarizing filters that each corresponds to (or are mapped to) a pixel of an image sensor. The filter horizontally polarizes light passing through the filter. The filter vertically polarizes light. One of the filters diagonally polarizes light, and another one of the filters circularly polarizes light.

Each of the filters include a wire grid having an MIM structure and one or more phase-modulating nanostructures. The horizontal and vertical dimensions of the phase-modulating nanostructures may be varied to achieve a desired amount of focusing. For example, the phase-modulating nanostructures may be generally square or circular, but having different horizontal and vertical dimensions (e.g., rectangular or ellipsoidal) depending upon the position of the nanostructure on the polarizing filter. The phase-modulating nanostructures of the circularly polarizing filter may be generally rectangular or ellipsoidal depending upon the position of the nanostructure on the polarizing filter.

The arrangement of the polarizing filters, in which the horizontally polarizing filter is in the upper-left corner of the polarizer, the vertically polarizing filter in the lower-right corner, the diagonally polarizing filter in the lower-left corner, and the circularly polarizing filter is in the upper-right corner, is an example arrangement and other arrangements are possible. In another example embodiment, two additional polarizing filters, such as an anti-diagonally polarizing filter and a circularly polarizing filter that would polarize light in the opposite circular direction from the circularly polarizing filter, may be included in the polarizer. Such an embodiment could also use two additional pixels. The polarizer may correspond to a super pixel.

In some embodiments, a multispectral and polarization sensing system enables the detection of both polarization and spectral information. The multispectral and polarization sensing system may include polarizing filters and spectral filters that provide on-chip simultaneous full Stokes polarization parameters (both linear and circular polarization) and multi/hyper spectral imaging. The multispectral and polarization sensing system may include a camera having a sensor or a photodiode. Furthermore, the multispectral and polarization sensing system may detect images from an array of pixels (2D signal information, referred to as an “image”) or light irradiated in one or more pixels (1D signal information, referred to as a “signal”). The multispectral and polarization sensing system may include polarizing and spectral filters. A 1D signal or 2D image captured by the camera having a sensor or a photodiode may be processed as a grayscale image and de-mosaiced. Additionally, the captured 1D signal or 2D image may be processed to generate corresponding multispectral linear and circularly polarized light that passes through the polarizing and spectral filters. For example, depending upon the particular polarizing and spectral filters that are used, the captured 1D signal or 2D image may generate multispectral horizontally polarized images, multispectral vertically polarized images, diagonally (45 degrees) polarized images, anti-diagonally polarized images, right-hand circularly polarized images, and left-hand circularly polarized images. Parameters determined from the linearly and circularly polarized images may be used to generate full Stokes parameters for the light of the image.

Further, the captured 1D signal or 2D image may be processed to generate non-polarized multispectral signals or images, and/or red (R), green (G) and blue (B) images. If the multispectral filters include filters for infrared (IR), multispectral IR images may be generated by the imaging system. Signals or images may be generated that indicate the degree of linear polarization (DoLP) and the degree of circular polarization (DoCP) may also be generated.

Accordingly, polarization information and spectral information may be generated based on the multispectral and polarization sensing system.

At least some or all of a light source and a detector may be combined to form a PPG sensor.

In some embodiments, a light source is provided. The lights source includes a diffractive element and/or collimation optics (a collimation optical element), polarization optics (a polarization optical element), and an LED/LD (additionally, other light emitting elements may be used (e.g., a vertical-cavity surface-emitting laser (VCSEL))) (an electromagnetic spectral emission source). The diffractive element and/or collimation optics may be combined as one element or separated as two separate elements, where the collimation optical element focuses or collimates an electromagnetic spectral emission into a narrow or tight beam (e.g., reduces the size (e.g., a diameter, circumference, or width) of the beam) to reduce diffusion, and the diffractive optical element may separate the electromagnetic spectral emission into a predetermined arrangement (e.g., lines, dots, or patterns). The diffractive element and/or collimation optics and the polarization optics may be combined with (or added to) the LED/LD so that the amount energy of light that is reflected back (e.g., reflected back from a user's skin) is increased. The LED/LD may emit light with frequencies in a visible range and/or a near infrared (NIR) spectrum.

The diffractive element and/or collimation optics may improve the efficiency input light by reducing the diverging aspects of the input light. The diffractive element and/or collimation optics may be configured to produce different types of outputs for the input light. For example, the diffractive element and/or collimation optics can diffract (or separate) the input light into lines, dots, a matrix pattern, or other predetermined arrangements. In this manner, the input light may be diffracted to a specific region or area causing that specific region or area to have a higher concentration of the light's energy. The polarization optics may control the input coefficient of the light.

In some embodiments, a detector is provided. Signals (e.g., light) having different spectrum interact differently with target molecules, biomarkers and health relevant parameters. For example, blue may be used for detecting antioxidant levels, like beta carotene; green may be used for pulse rate monitoring because it is less influenced by the tissue and vein region than other colors and/or spectra. Red and IR may be used by determining a difference in absorbance at those two frequencies (a red frequency versus an IR frequency) between oxygenated and unoxygenated hemoglobin. Using the difference in these two frequencies allows the concentration of oxyhemoglobin to be calculated (e.g., the red LED may be at the frequency where oxyhemoglobin and hemoglobin have identical absorbances).

The detector includes a polarization filter, a VIS/NIR filter, and a PD/APD/SPAD. The polarization filter may include a polarization filter array. The VIS/NIR filter may be an electromagnetic spectrum filter and may include a color filter either organic or inorganic, a nanostructured color filter, a narrow band filter, distributed Bragg filters or a broadband filter. Some or all of the filters and/or components in the VIS/NIR filter may be made of a stack of semiconductor(s) and/or oxides/nitride. For example, each of the filters included in the VIS/NIR filter may be included in a stack of semiconductors but for the color filter. In addition, the VIS/NIR filter may detect signals that penetrate more deeply into the skin due to less scattering.

The PD/APD/SPAD may include a sensor, which allows the detector to measure both spectral light information and polarized light information. PD, APD, and SPAD sensors may have different sensitives, which may improve (e.g., reduce) SNR for detecting signals for different applications. For example, SPAD may work well with a laser input signal, where PD and APD may work better with an LED input signal. In addition, different linear polarized light may interact differently with different bodily materials (e.g., fat, blood, or arteries). Circular polarized light may interact differently with molecules like skin cancer melanoma, antioxidants, triglyceride and so on.

Accordingly, the detector may be sensitive to polarization calibration and spectrum calibration. In addition, the polarization filter may include aluminum (Al), titanium oxide (TiO₂), aluminum oxide (Al₂O₃), tungsten (W), silicon oxide (SiO₂), silicone (Si), silicon nitride (Si₃N₄), and amorphous silicon (a-Si).

In some embodiments, a PPG sensor includes a light source and a detector.

The light source includes four light emitting elements. Each of the light emitting elements may correspond to different spectrums (e.g., different colors). Light emitting elements having different spectral characteristics advantageously behave differently when they come into contact with an object (e.g., a user's skin). This advantage may be used for single color pixel targeting by light emitting elements emitting wavelengths using on-chip polarizer filters. The light emitted from the light emitting elements may each have a narrow bandwidth (e.g., a single color) or a wide bandwidth (e.g., covering visible light all the way to infrared (IR) light).

The detector is made up of a number of super pixels in proximity to the light source. Each of the super pixels in the detector may include features of the polarizer. That is, each of the super pixels in the detector may include four distinct pixels (e.g., portions of the super pixel) that are each capable of filtering light differently, such as how light is filtered by polarizing filters (corresponding to pixels), to collect light polarization information. Additionally, each super pixel may include more than four pixels (e.g., six) to collect spectral information and light polarization information.

Furthermore, each super pixel may be designed to detect light of a predetermined frequency (corresponding to a spectrum of one of the four light emitting elements). That is, a first pattern type may correspond to a first spectrum, a second pattern type may correspond to a second spectrum, a third pattern type may correspond to a third spectrum, and a fourth pattern type may correspond to a fourth spectrum.

Additionally, the distribution (e.g., physical arrangement) of the super pixels on the left side to the light source is shown to be vertical (e.g., a vertical distribution of 3 super pixels). The distribution of the super pixels may enable the PPG sensor to determine a depth at which the detected light penetrates, as well as other characteristics based on the distribution. For example, the super pixels having a first pattern type corresponding to the first spectrum may identify light output from the light emitting element having the first pattern type corresponding to the first spectrum of the light source based on an angle of the detected light at each of the super pixels. Since the light having the first spectrum is output at a particular area of the light source, the super pixels capable of detecting the light having the first spectrum may be arranged so that some of the super pixels detect the light having the first spectrum at different angles than others. The angle information may be used to determine the depth at which the light penetrates. Additionally, a time difference from which the light is detected by a first super pixel at a first location and a second super pixel at a second location may be used to determine the angle information.

The arrangement of the super pixels and their corresponding light emitting elements (e.g., super pixels and light emitting element for the first spectrum having the first pattern, super pixels and light emitting element for the second spectrum having the second pattern, etc.) are not limited to that which is shown. Many different alternative arrangements are possible which may be capable of detecting different characteristics of the light due to the alternative arrangements. Thus, the light source and detector arrangements of the PPG sensors can be designed to satisfy particular light detection characteristics that are sought by the design.

Data produced by the PPG sensors may be processed in an application processor on the same platform (e.g., a chip) or in the cloud.

In some embodiments, the PPG sensor includes a light source and a detector.

In some embodiments, the light source includes four light emitting elements having different light spectrums (corresponding to the four different patterns). In some embodiments, each set of super pixels corresponding to each light spectrum may be oriented as straight lines facing outward from the light source. The super pixels may form lines at approximately 0, 90, 180, and 270 degrees, however other arrangements are possible depending on the design requirements of the PPG sensor. For example, super pixel lines may be oriented at 45, 135, 225, and 315 degrees. In addition, multiple sets of super pixels may be added, thereby increasing the total number of super pixels. The super pixels are each made up of at least four pixels to detect different polarity characteristics of the light emitted from the light source.

In some embodiments some of the super pixels are arranged farther away from the light source than in other embodiments (where each of the super pixels are arranged around the light source), and the super pixels arranged farther away may be able to detect larger angles of the reflected light emitting from the light source. Generally, larger angles are associated with larger depths. However, for the super pixels that are arranged far away from the light source, sensitivity of the detected light may be lower as compared to super pixels arranged closer to the light source.

In some embodiments, the PPG sensor includes a light source and a detector.

The super pixels of the detector each include a number of pixels (e.g., four). For example, the super pixels may include a λ (generally corresponding to a wavelength), a variable (e.g., “x”, “y”, or “z”), and a number. The super pixels are incrementally listed. For example, a top row of super pixels are incrementally listed: “λx1”, λx2” . . . “λxn”, where n represent the total number of super pixels in this grouping. The variable “x” may correspond to a spectral range (e.g., a spectral range for blue light). Thus, the combination of the “λx1”, λx2” . . . “λxn” super pixels may be very sensitive to detecting particular spectral ranges of the blue light. Furthermore, the “y” variable may correspond to a different spectral range (e.g., a spectral range for green light), and the “z” variable may also correspond to a different spectral range (e.g., a spectral range for red light). Accordingly, each set of super pixels may be designed to precisely capture and identify light having a wide spectral range.

Since the super pixels of the detector are each capable of detecting many different spectrums of lights, the light emitting elements in the light source should be arranged to ensure that each light emitting element is capable of emitting (powerful or proximate enough to emit) light to the pixels at each super pixel for detection.

In some embodiments, the PPG sensor includes a light source and a detector.

In some embodiments, a plurality of wide spectral range super pixels are provided in an array like pattern in the detector on each side of the light source. The array like pattern may improve the accuracy of what particular area of the detector light is detected at.

The present disclosure also relates to various embodiments of a flowchart for biosignal detection and analysis.

In some embodiments, at one step, input light is collimated or diffused. Accordingly, the input light can be separated into lines, dots, a matrix pattern, or other predetermined arrangements. In this manner, the input light may be diffracted to a specific region or area causing that specific region or area to have a higher concentration of the light's energy.

At another step, polarized light (e.g., an electromagnetic spectrum emission) is emitted. For example, the input light that is collimated or diffused may be emitted by a light source in an electronic device.

At another step, the emitted polarized light is reflected off of a surface and collected. For example, as described above, a sensor featuring four pixels included in an electronic device may collect the reflected polarized light. The surface may be living tissue and may include multiple surfaces therein. For example, the polarized light may be reflected off of skin, with some of the light being reflected off an epidermis layer of the skin and some of the light being reflected off of a dermis layer of the skin. In addition, some of the polarized light may be reflected by blood arteries, and molecules included therein.

At a step, multispectral information of the reflected polarized light is collected. This may include polarized light having different wavelengths or frequencies. In addition, polarized light within predefined wavelength or frequency ranges may be collected.

At a step, a polarized light component of individual pixels from each of the spectrums is determined.

At a step, the Stokes parameters (e.g., S₀, S₁, S₂, and S₃) are calculated, and all six polarization states (I_{H}, I_{V}, I_{D}, I_{L}, I_{A}, and I_{R}) may be determined. These values may be used to identify particular molecules or parameters of the surface which the polarized light is reflected.

At a step, at least one additional signal is calculated based on the Stokes parameters, such as depolarization state information, retardance information, and diattenuation information.

At a step, the additional signal is tracked over time or wavelength. That is, a first intensity or amplitude of the additional signal may be determined at a first time point, a second intensity or amplitude of the additional signal may be determined at a second time point after the first time point, etc.

At a step, the additional signal is analyzed and measured to reveal physiological parameters (e.g., heart-rate information, respiratory rate information, hypertension signatures, red blood cell concentration information, blood saturation information, a continuous blood pressure, pulse rate information, a pulse pressure, cardiovascular conditions, stroke volume information, cardiac output information, a one lead electrocardiogram (ECG), a systematic vascular resistance, cardiac index, a mean arterial pressure, antioxidants, melanoma information, triglyceride information, cholesterol information, and beta carotene information) related to the surface from which the polarized light is reflected.

A relationship among parameters may be used to process a signal calculated based on reflected polarized light, according to an embodiment.

In some embodiments, a Mueller matrix M may be applied to the Stokes parameters to obtain at least one of information related to depolarization M_{Δ}, retardance M_{R} and/or diattenuation M_{D}.

Depolarization MA may refer to the magnitude (or amount) of polarization and can include linear depolarization information Δ_{L}, circular depolarization information Δ_{C} and/or total depolarization information Δ.

Retardance M_{R} may refer to the magnitude (or amount) of an optical path difference (e.g., a phase shift) experienced by polarized light. The retardance MR may include a linear retardance matrix M_{RL} to identify anisotropy information, including a linear retardance δ and an orientation θ to identify a degree and angle of polarization. Retardance M_{R} may also be calculated with circular polarization using a circular retardance matrix M_{ψ} to identify optical activity w (e.g., the direction of the circular polarization (e.g., clockwise or counterclockwise)).

Diattenuation M_{D} may refer to the intensity of the signal with respect to a polarization state. Accordingly, diattenuation M_{D} may indicate one or more ratios of intensities of different polarization states.

According to an embodiment, a method for biosignal detection and analysis may use polarized microscopy to improve SNR and measure antioxidants. For example, S₀ may represent the intensity of the light (polarized+unpolarized); S₁ may represent the intensity of linear horizontal or vertical polarization; S₂ may represent the intensity of linear +45° or −45° polarization; and S₃ may represent the intensity of right or left circular polarization. Accordingly, using the Stokes parameters S₀-S₃, a signal may be calculated based on reflected polarized light to identify, for example, characteristics of a target molecule that is beneath a surface having a defined thickness.

Different types of polarized light (e.g., linear polarized light, circular polarized light) can be used to reveal different characteristics about the surface from which it is reflected. For example, unpolarized light and polarized light having intensities of I_{0°} and I_{45°} and I_{90°} may each be emitted and reflected off of a surface. The unpolarized light typically does not penetrate surfaces well, and therefore produces a relatively low quality SNR. I90° polarized light may penetrate surfaces better than the unpolarized light, thereby having a better SNR. Additionally, if I_{0°} and I_{90°} and I_{90°} polarized light are each emitted, reflected, and measured, then the resulting SNR may be better than the I_{90°} polarized light, since Stokes parameters for each of the different types of polarized light may be applied.

In addition, different molecules may be sensitive to different types of polarized light. For example, a beta carotene molecule is highly sensitive to circular polarization and, therefore, beta carotene can be detected by comparing the DoLP with the DoCP. In other words, since beta carotene is highly sensitive to circular polarization and is not sensitive to linear polarization, the DoCP may reveal the existence of beta carotene molecules by detecting the circular polarization intensity at different wavelengths. For example, a peak to trough (valley) ratio of the DoCP may be calculated for a predefined wavelength range to assess the likelihood that beta carotene is present. Generally, the larger the wavelength range, the more accurate the detection will be.

Additionally, the different types of polarized light can be used to reveal cardiovascular signatures and information about blood, fat, arteries, and tendons.

In some embodiments, a plurality of types of polarized light are emitted towards a blood artery. Since the different types of polarized light feature different characteristics, detecting the different types of polarized light may reveal information necessary to calculate biosignals at different depths. For example, linear polarized light having one angle of polarization may be reflected off of an outer surface of the artery wall, linear polarized light having two angles of polarization may penetrate the artery wall and may be reflected off of a first inner surface of the artery wall, and linear polarized light having four angles of polarization may penetrate even further and be reflected off of a second inner surface of the artery wall.

After detection, Stokes parameters may be calculated for each of the reflected and detected types of polarized light to determine biosignals for the respective depths of penetration of the polarized light. The polarized light may be used, for example, to detect a wall-to lumen ratio of retinal arterioles as a tool to assess vascular changes, since a thickness of the artery wall relative to the thickness of the lumen may indicate health-related information.

Thus, as described above, methods and systems are provided for using a polarized light source and multispectral full Stokes polarization PPG sensors to identify biosignals and other health related information. In addition, simultaneously modulating the phase and polarization of the light may improve SBR and field of view (FOV) properties. Accordingly, spectral sensitive physiological parameters and molecular information (e.g., antioxidant information, triglyceride information, cholesterol information, etc.) can be identified based on detecting the different types of polarized light reflected off of a surface of a living tissue (e.g., skin).

Any task(s), portion(s), sub-portion(s), component(s), or sub-component(s) (or any combination or sub-combination thereof) of the above-described methods and systems may be applied, incorporated, or otherwise utilized in connection with the following embodiments of the present disclosure.

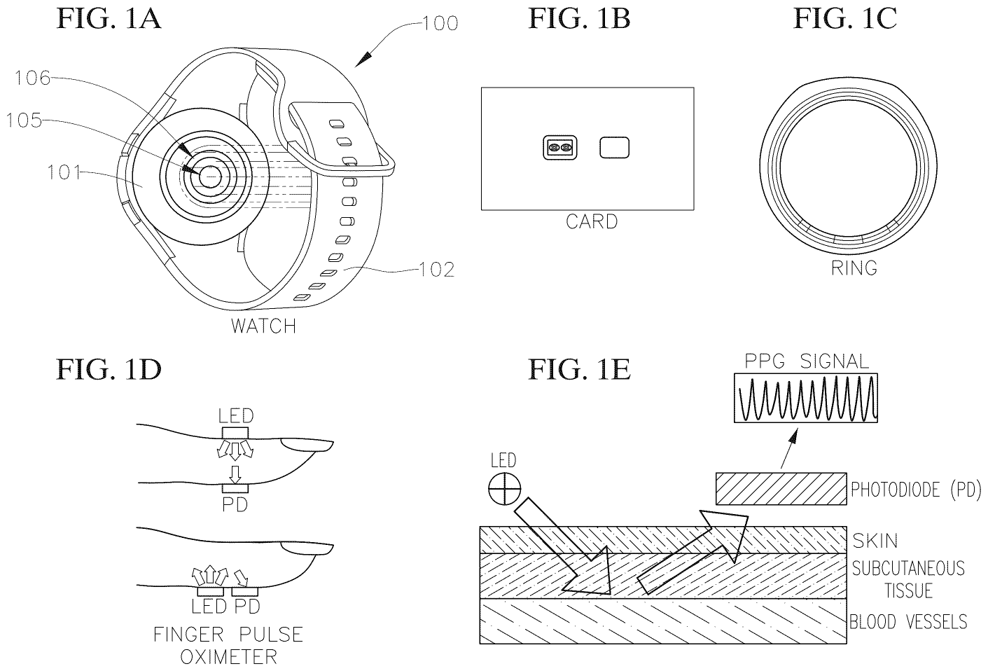

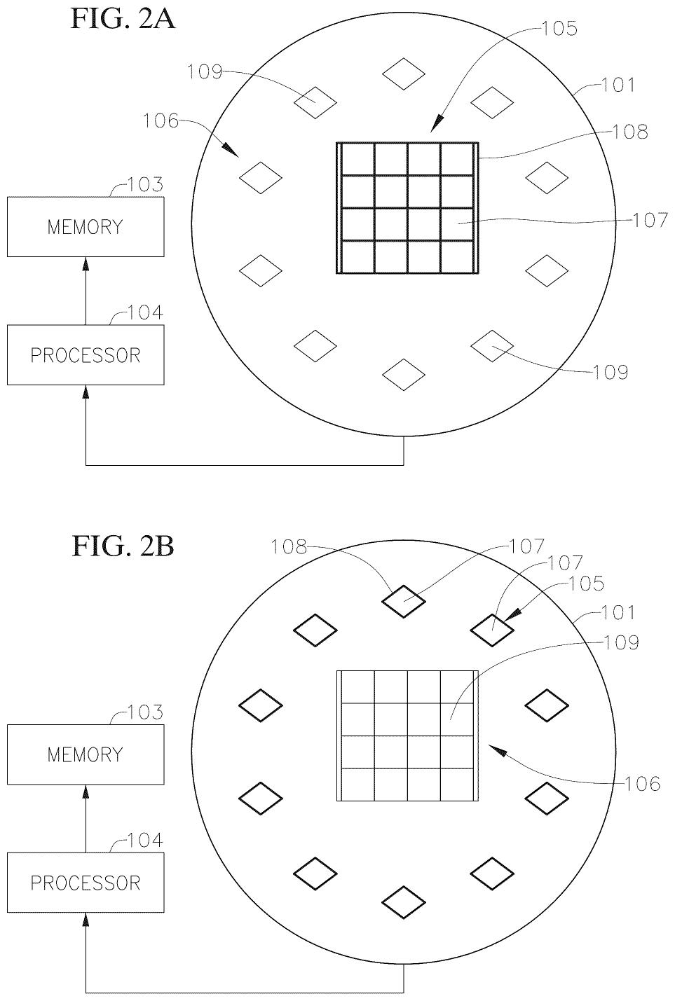

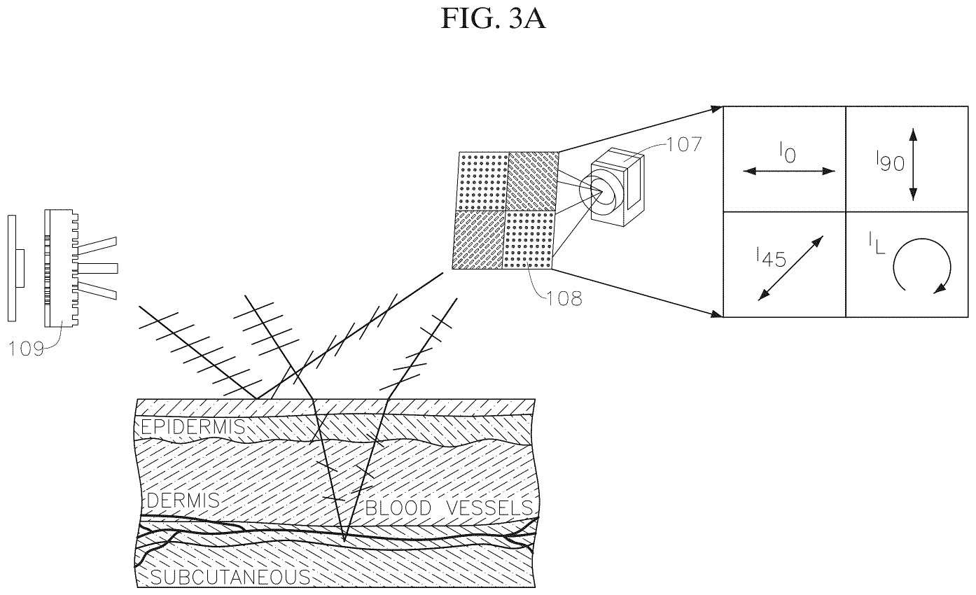

With reference now to FIGS. 1A-2B, a wearable biometric measurement device 100 configured to perform photoplethysmography (PPG) according to one embodiment of the present disclosure includes a housing 101 and a strap 102 (e.g., a wristband) coupled to the housing 101. In one or more embodiments, the wearable biometric measurement device 100 may be any suitable type or kind of wearable device, such as a watch (shown in FIG. 1A), a patch (e.g., a skin patch), an earbud, AR/VR goggles, a card (shown in FIG. 1B), a ring (shown in FIG. 1C), or a finger pulse oximeter (shown in FIG. 1D). In the illustrated embodiment, the housing 101 includes a non-volatile memory device 103, a processor 104 in electronic communication with the non-volatile memory device 103, a photodiode array 105, and light-emitting diode (LED) array 106. In the illustrated embodiment, the photodiode array 105 includes a plurality of photodiodes 107 and a plurality of polarization filters 108 covering the plurality of photodiodes 107. In one or more embodiments, all of the polarization filters 108 may be the same or substantially the same (e.g., all of the polarization filters 108 may be linear 0° polarization filters) or the polarization filters 108 may be a combination of two or more different polarization filters (e.g., the polarization filters 108 may include two or more different polarization filters selected from linear filters, such as 0°, 45°, 90°, or 135° linear polarization filters, and circular polarization filters (right or left)). Additionally, in the illustrated embodiment, the LED array 106 includes a plurality of light-emitting diodes (LEDs) 109. FIG. 1E is a schematic view depicting light from the LED array 106 reflecting off a vessel below subcutaneous tissue underneath the user's skin and being captured by the photodiode array 105, which generates a PPG signal.

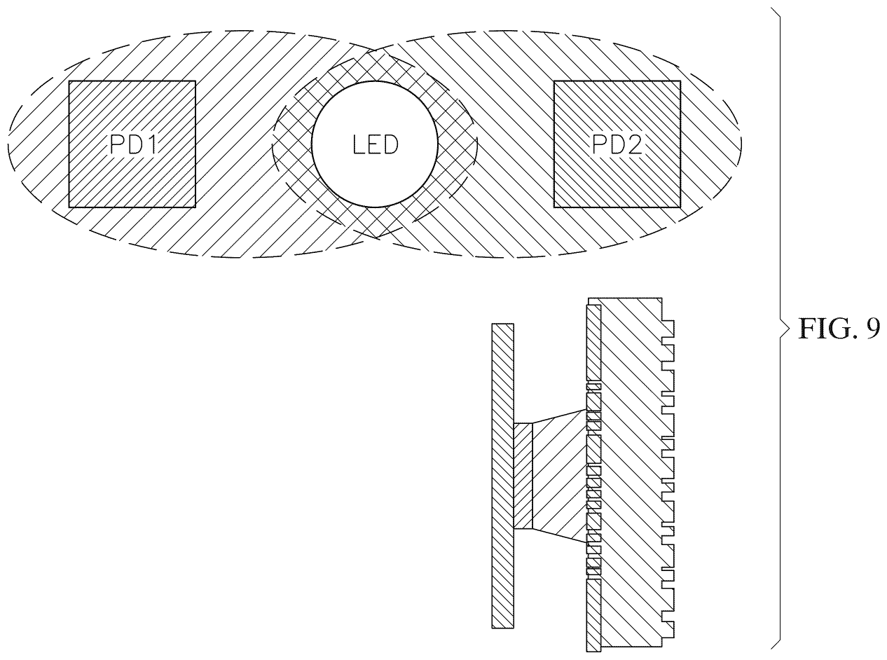

The LEDs 109 of the LED array 106 may be arranged in any suitable configuration proximate to the photodiodes 107 of the photodiode array 105. For instance, in the embodiment illustrated in FIG. 2A, the photodiodes 107 are arranged in a rectangular grid and the LEDs 109 are arranged in an annular (e.g., circular) configuration around (surrounding) the photodiode array 105. In the embodiment illustrated in FIG. 2B, the LEDs 109 are arranged in a rectangular grid and the photodiodes 107 are arranged in an annular (e.g., circular) configuration around (surrounding) the LED array 106. In one or more embodiments, the number of LEDs 109 in the LED array 106 may be in a range from 2 LEDs to 12 LEDs. The one or more LEDs 109 and the one or more photodiodes 107 may be arranged in any other suitable arrangement depending on the type of biometric measurement device in which they are incorporated, such as a card, a ring, or a patch. In one or more embodiments, the one or more LEDs 109 and the one or more photodiodes 107 may be arranged in a linear arrangement (e.g., one LED 109 may be arranged in the center along a linear direction and one or more photodiodes 107 may be arranged on opposite sides of the LED 109 along the linear direction). For instance, FIG. 9 depicts an embodiment in which an LED and a pair of photodiodes (“PD1” and “PD2”) on opposite sides of the LED are arranged in a linear array. Additionally, in one or more embodiments, the LEDs 109 in the LED array 106 may each be configured to emit light have the same (or substantially the same) wavelength. In one or more embodiments, the LEDs 109 in the LED array 106 may be configured to emit light having two or more different wavelengths (e.g., a first LED 109(1) in the LED array 106 may be configured to emit light having a first wavelength and a second LED 109(2) in the LED array 106 may be configured to emit light having a second wavelength different than the first wavelength). The LEDs 109 may be configured to emit polarized light or unpolarized light. In one or more embodiments, the emissions from the one or more LEDs 109 can be controlled by utilizing optical diffraction filters on the one or more LEDs 109 to increase the light collection by the photodiodes 107. Additionally, in one or more embodiments, one or more metaphotonic optical filters may be provided on the one or more LEDs 109 to improve the light emission from the one or more LEDs 109. Metaphotonic filters are described in U.S. Pat. No. 11,841,522 and U.S. Patent Application Publication Nos. 2024/0065566 and 2023/0358936, the entire contents of which are incorporated herein by reference.

The non-volatile memory device 103 includes computer-readable instructions (i.e., software code) which, when executed by the processor 104, cause the wearable biometric measurement device 100 to perform photoplethysmography (PPG). In one or more embodiments, the computer-readable instructions, when executed by the processor 104, cause the LEDs 109 to emit light. For instance, in one or more embodiments, the computer-readable instructions, when executed by the processor 104, may cause two or more of the LEDs 109 to emit light simultaneously (i.e., the computer-readable instructions, when executed by the processor 104, cause two or more of the LEDs 109 to turn on in parallel). In one or more embodiments, the computer-readable instructions, when executed by the processor 104, cause two or more of the LEDs 109 to turn on sequentially. For instance, in one or more embodiments, the computer-readable instructions, when executed by the processor 104, cause a first LED 109 of the LED array 106 to turn on and emit light at a first time t₁ and cause a second LED 109 of the LED array 106 to turn on and emit light at a second time t₂ after the first time t₁.

In one or more embodiments, the computer-readable instructions, when executed by the processor 104, cause the processor 104 to receive the signals output by the photodiodes 107 in response to the photodiodes 107 receiving the reflected light signal (i.e., the portion of the light waveform that was emitted from the LED(s) 109, reflected off of the user's blood vessel, and reached at least one of the photodiodes 107).

In one or more embodiments, the computer-readable instructions, when executed by the processor 104, cause the processor 104 to calculate or determine a physiological parameter according to Equation 15 as follows:

$${S}_{t} = f \frac{AC ( {λ}_{n , P} )}{DC ( {λ}_{n , P )}} \qquad ( Equation 15 )$$

where Sₜ is a signal at a time t; ƒ is a function; AC(λ_{n,P}) is the pulsatile (AC) component of the reflected waveform collected with light having a wavelength λₙ and a polarization state P; DC(λ_{n,P)} is the static (DC) component of the reflected waveform collected with light having a wavelength λₙ and a polarization state P; and n is the target LED and where the total number of LEDs is N and n goes from 1, 2, 3, . . . N. In one or more embodiments, the polarization state P may be 0°, 30°, 45°, 60°, 90°, 120°, 135°, 150°, R, or L.

In one or more embodiments, the computer-readable instructions, when executed by the processor 104, cause the processor 104 to calculate or determine the oxygen saturation SpO₂ of the user according to Equation 16 as follows:

$${SpO}_{2} = α = β \cdot R \qquad ( Equation 16 )$$

where R is calculated according to Equation 17 as follows:

$$R = ( {AC}_{rms , λ1} / {DC}_{rms , λ1} ) / ( {AC}_{rms , λ2} / {DC}_{rms , λ2} ) \qquad ( Equation 17 )$$

where AC_{rms,λ1} is the root-mean-square of the pulsatile (AC) component of the reflected waveform collected with light having a wavelength λ₁; DC_{λ1} is the static (DC) component of the reflected waveform collected with light having a wavelength λ₁; AC_{rms,λ2} is the root-mean-square of the pulsatile (AC) component of the reflected waveform collected with light having a wavelength λ₂; and DC_{λ2} is the static (DC) component of the reflected waveform collected with light having a wavelength λ₂.

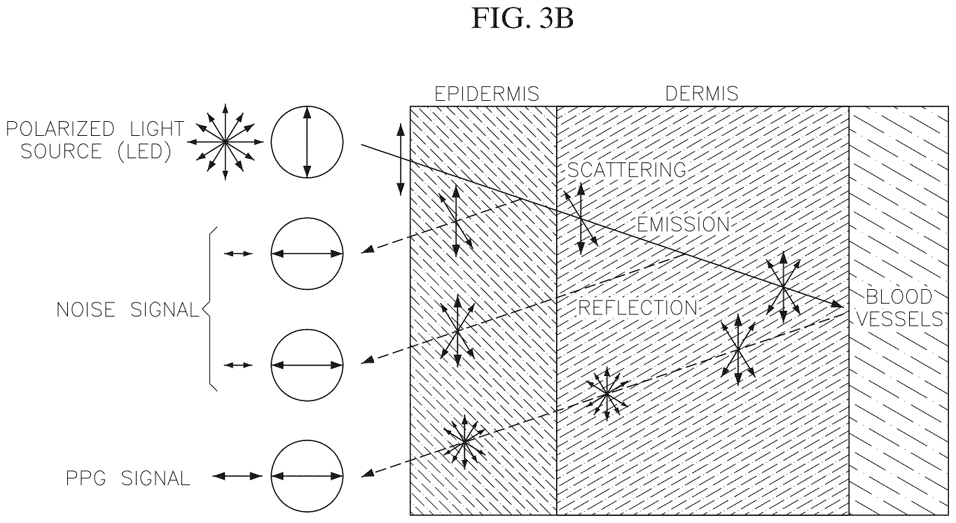

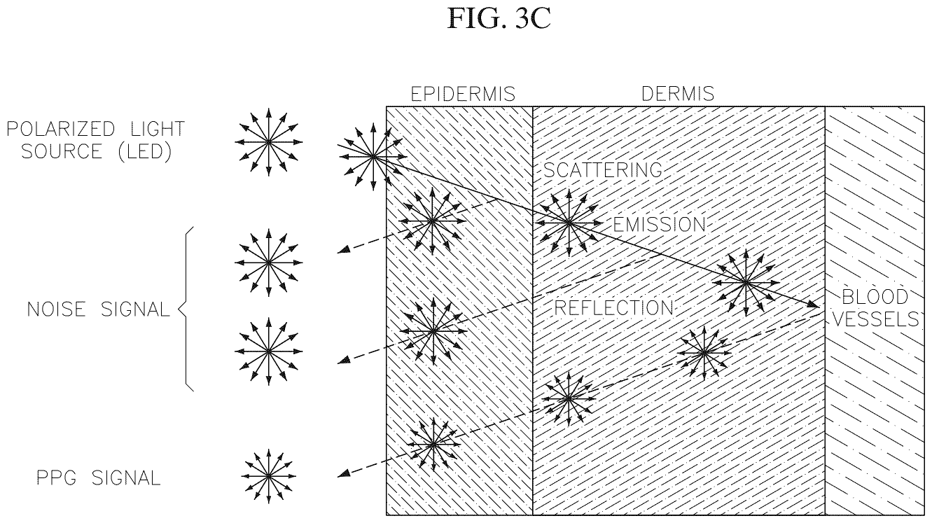

FIGS. 3A-3B are schematic diagrams depicting operation of the wearable biometric measurement device 100 in which polarized light is emitted from one of the LEDs 109, reflected off a user's blood vessel, and received at one of the photodiodes 107 with the polarization filter 108. FIG. 3C is a comparative example of a related art device in which non-polarized light is emitted from an LED, reflected off a user's blood vessel, and received at a photodiode without a polarizer. As illustrated in FIGS. 3B and 3C, the use of polarized light (e.g., polarized light emitted from the LED(s) 109 and/or the polarization filter 108 on the photodiode(s) 107) reduces specular reflection off of the user's blood vessel, increases the signal-to-noise ratio, and thereby improves the perfusion index (PI), which may be utilized to calculate a physiological parameter of the user.

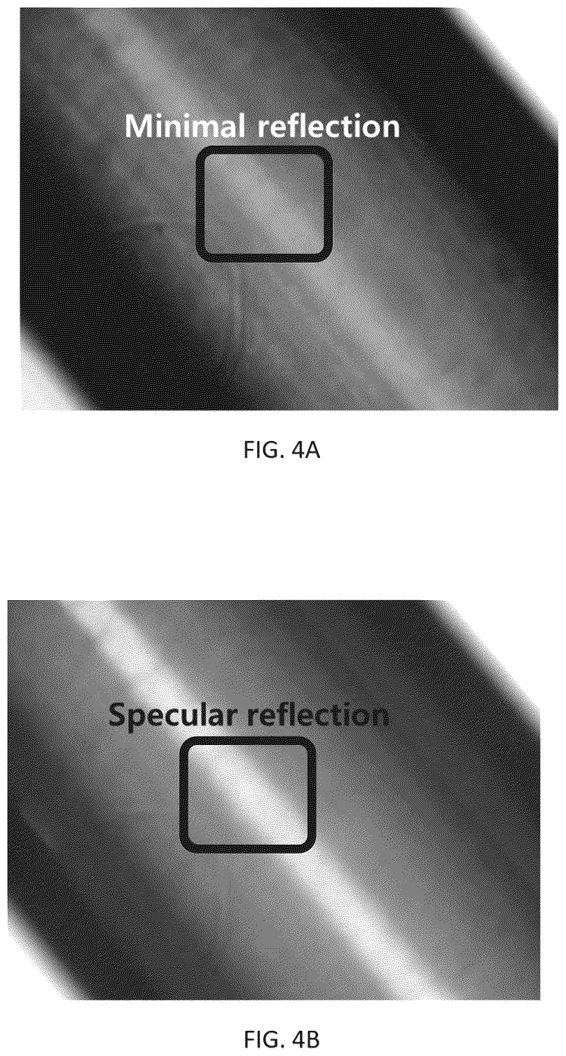

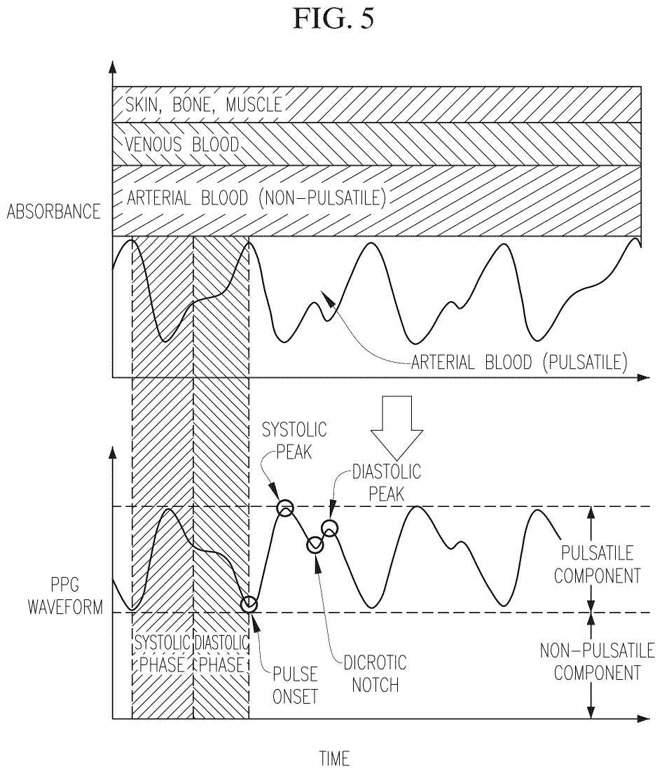

FIG. 4A is a photograph of the specular reflection off a device/tube mimicking a user's blood vessel when the device/tube mimicking the user's blood vessel is irradiated with polarized light from one of the LEDs 109 of the LED array 106. FIG. 4B is a comparative example photograph of the specular reflection off a device/tube mimicking a user's blood vessel when the device/tube mimicking the user's blood vessel is irradiated with non-polarized light. As shown in FIGS. 4A-4B, utilizing polarized light to irradiate the user's blood vessel results in comparatively less specular reflection, which would otherwise create noise in the reflected waveform signal. For instance, in one or more embodiments, utilizing a polarization filter reduced specular reflection by approximately 40% compared to not using a polarization filter.

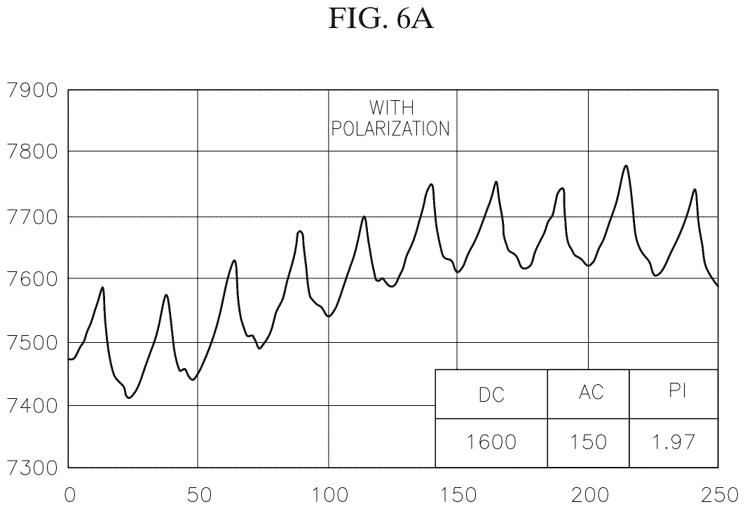

FIG. 5 is a graph depicting the waveform reflected off a user's blood vessel when irradiated with light from one of the LEDs 109 of the LED array 106. As illustrated in FIG. 5, the waveform includes a pulsatile (AC) component and a static (DC) component. The pulsatile (AC) component of the reflected waveform is due to the pumping action of the heart and the resultant change in blood volume in the blood vessel. The pulsatile component of the reflected waveform is due to the residual blood in the blood vessel as well as reflection from skin, tissue, and bones.

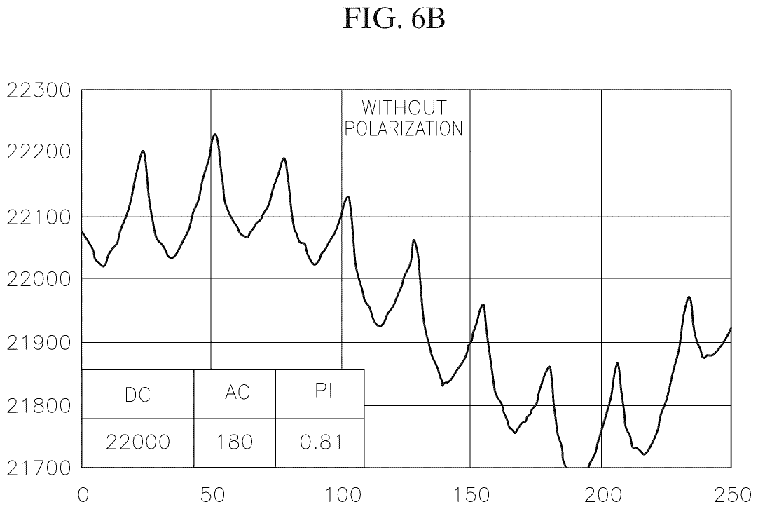

FIGS. 6A-6B are graphs depicting the perfusion index (PI) of the reflected waveform when the blood vessel is irradiated with polarized light and non-polarized light, respectively. As illustrated in FIG. 6A, the photodiode with the polarization filter had a perfusion index of 1.97, with an AC component of 15,000 and a DC component of the reflected waveform of 7,600 (i.e., 15,000/7,600=1.97). In contrast, as illustrated in FIG. 6B, the photodiode without a polarization filter had a perfusion index of 0.81, with an AC component of 18,000 and a DC component of the reflected waveform of 22,000 (i.e., 18,000/22,000=0.81).

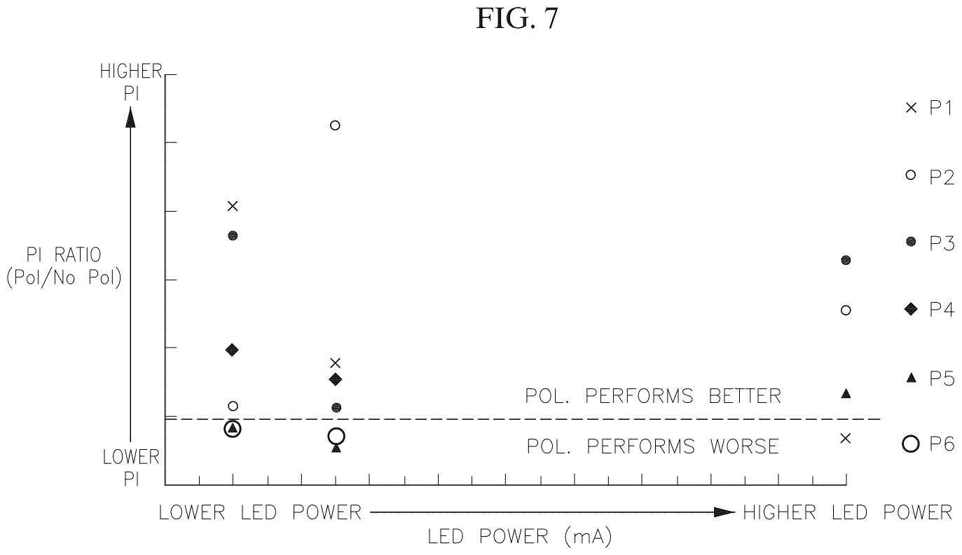

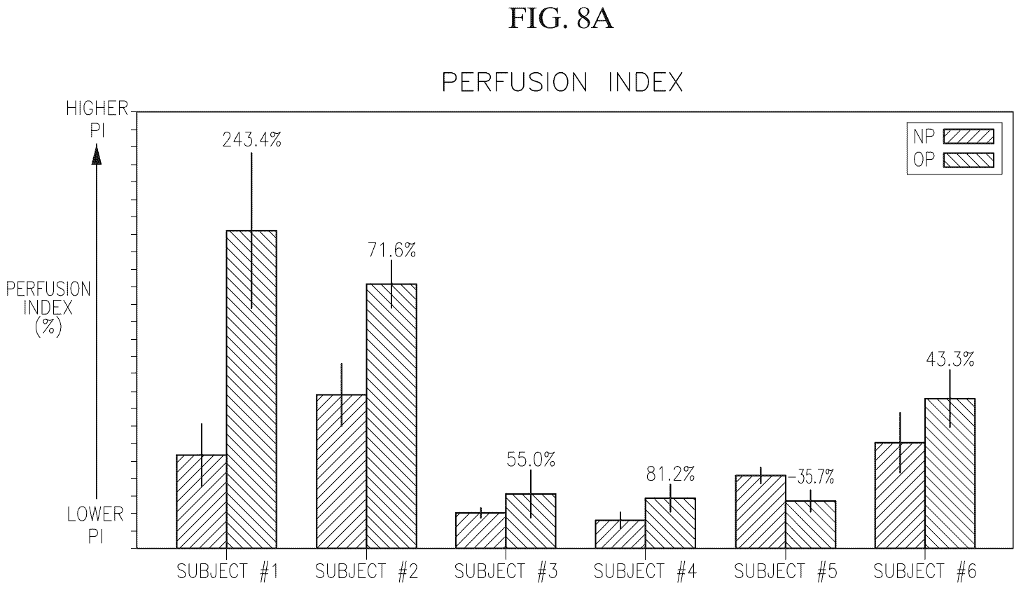

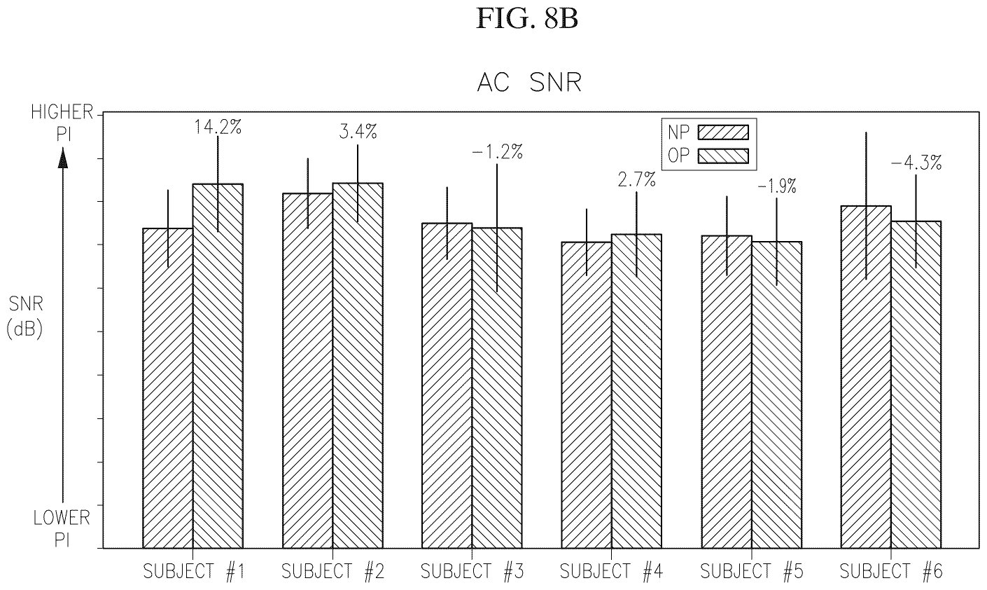

FIG. 7 is a graph depicting the perfusion index (PI) ratio as a function of LED power according to one embodiment of the present disclosure. The PI ratio was calculated for each of six different users, labeled P1, P2, P3, P4, P5, and P6 In FIG. 7, the PI ratio is a ratio of the perfusion index (i.e., AC/DC) of the wearable biometric measurement device 100 according to one embodiment of the present disclosure that utilizes a polarization filter on the photodiode to the perfusion index of a photodiode without a polarization filter. In FIG. 7, a PI ratio of 1.0 indicates that the photodiode with the polarization filter and the photodiode without the polarization filter performed equally well; a PI ratio less than 1.0 indicates that the photodiode without the polarization filter performed better than the photodiode with the polarization filter; and a PI index greater than 1.0 indicates that the photodiode with the polarization filter performed better than the photodiode without the polarization filter. As illustrated in FIG. 7, in the majority of the cases, the photodiode with the polarization filter outperformed the photodiode without the polarization filter. For instance, in many instances, the perfusion index of the photodiode with the polarization filter is approximately 2 times to approximately 4 times greater than the perfusion index of the photodiode without the polarization filter. FIG. 8A depicts the perfusion index (%) of the photodiode with the polarization filter (“OP”) and without the polarization filter (“NP”) for each of six different human subjects (Subject #1; Subject #2; Subject #3; Subject #4; Subject #5; Subject #6) with different skin tones. FIG. 8B depicts the AC signal-to-noise ratio (SNR) of the photodiode with the polarization filter (“OP”) and without the polarization filter (“NP”) for each of the six human subjects with different skin tones. As shown in FIGS. 8A-8B, the wearable biometric measurement device 100 including the polarization filter can improve the quality of signal and therefore provide more accurate measurements for measuring the physiological parameter(s) of users having a variety of different skin tones. Accordingly, the wearable biometric measurement device 100 of the present disclosure is able to avoid or overcome the well-known problem that related art optical healthcare sensors perform poorly when collecting light-based vital-sign measurements from users with a higher concentration of melanin in the skin, as described in H. Bridger, “Skin Tone and Pulse Oximetry,” available online at https://hms.harvard.edu/news/skin-tone-pulse-oximetry, and R. Al-Halawani, et at., “A review of the Effect of Skin Pigmentation on Pulse Oximeter Accuracy,” available online at https://www.ncbi.nlm.nih.gov/pmc/articles/PMC10391744/#:˜:text=SpO2%20was%20less%20accurate,in%20Black%20than%20White%20pa.

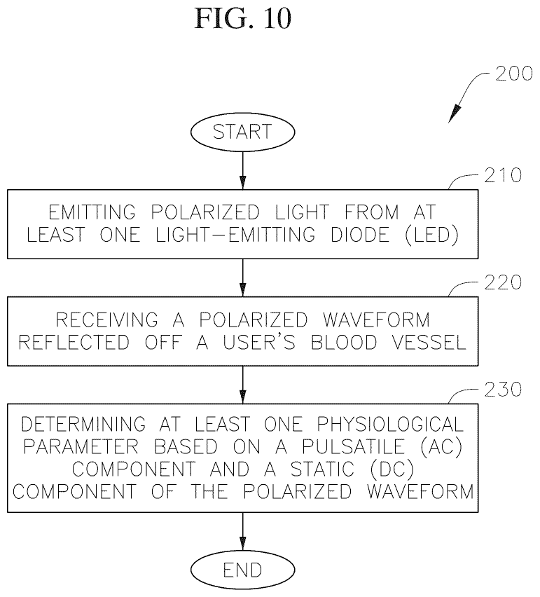

FIG. 10 is a flowchart illustrating tasks of a method 200 of performing photoplethysmography according to one embodiment of the present disclosure. In the illustrated embodiment, the method 200 includes a task 210 of emitting light from at least one light-emitting diode of a number of light-emitting diodes arranged in a light-emitting diode (LED) array. The LED array may have any suitable configuration, such as an annular arrangement (e.g., as illustrated in FIG. 2A) or a grid arrangement (e.g., as illustrated in FIG. 2B). In one or more embodiments, the light-emitting diodes may be configured to emit the same (or substantially the same) wavelength of light. In one or more embodiments, the light-emitting diodes may be configured to emit two or more different frequencies of light.

In the illustrated embodiment, the method 200 also includes a task 220 of receiving a polarized waveform from a portion of the light emitted by the at least one light-emitting diode in task 210 after it reflects off a user's blood vessel (i.e., the task includes receiving a polarized waveform from reflected light). In task 220, the polarized waveform is received by at least one photodiode of a number of photodiodes arranged in a photodiode array. The photodiode array may have any suitable configuration, such as a grid arrangement (e.g., as illustrated in FIG. 2A) or an annular arrangement (e.g., as illustrated in FIG. 2B). In one or more embodiments, the photodiode includes a polarization filter configured to polarize the reflected light. In one or more embodiments, all of the polarization filters may be the same or substantially the same (e.g., all of the polarization filters may be linear 0° polarization filters) or the polarization filters may be a combination of two or more different polarization filters (e.g., the polarization filters may include two or more different polarization filters selected from linear filters, such as 0°, 45°, 90°, or 135° linear polarization filters, and circular polarization filters (right or left)).

In the illustrated embodiment, the method 200 also includes a task 230 of determining at least one physiological parameter based on a pulsatile (AC) component and a static (DC) component of the polarized waveform received in task 220. For instance, in one or more embodiments, the task 230 may include calculating the physiological parameter according to Equation 15 above. In one or more embodiments, the task 230 may include calculating the oxygen saturation SpO₂ of the user according to Equation 16 and Equation 17 above. As described above, the use of polarized light (e.g., by emitting polarized light in task 210 and/or collecting the reflected waveform with a polarization filter in task 230) is configured to reduce specular reflection off of the user's blood vessel, which reduces signal artifacts and improves the signal-to-noise ratio and therefore improves the accuracy of the determination of the physiological parameter in task 230.

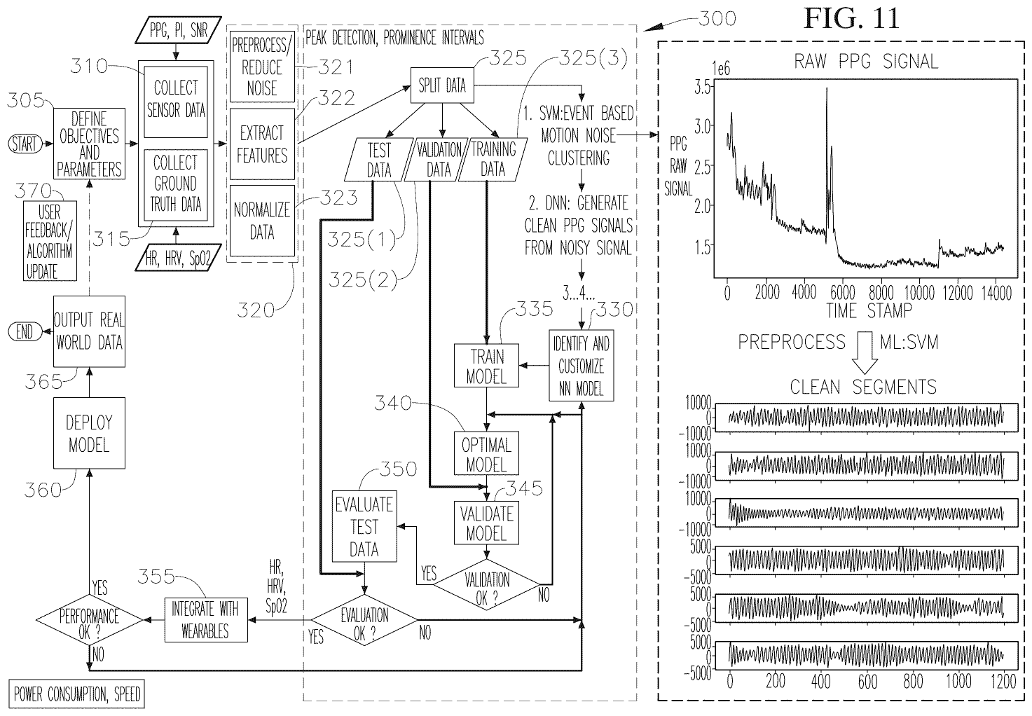

FIG. 11 is a flowchart depicting tasks of an artificial intelligence (AI)-based method 300 of performing photoplethysmography (PPG) according to one embodiment of the present disclosure. The method 300 is configured to process a raw photoplethysmography signal and convert or transform it into cleaner data utilizing machine learning. In the illustrated embodiment, the method 300 includes a task 305 of defining the relevant objectives and parameters, such as PPG signal-to-noise ratio (SNR) being greater than approximately 20%, heart rate (HR) mean absolute error (MAE) being less than approximately 5 bpm, heart rate variability (HRV) mean absolute error (MAE) being less than approximately 10 ms, and/or blood oxygen saturation (SpO₂) root mean squared error (RMSE) being less than approximately 5%.

The method also includes a task 310 of collecting sensor data (e.g., PPG, PL, SNR) and a task 315 of collecting ground truth data (e.g., HR, HRV, SpO₂). The method 300 further includes a task 320 of modifying the data, such as a sub-task 321 of preprocessing the data (e.g., reducing noise), a sub-task 322 of extracting features from the data, and/or a sub-task 323 of normalizing the data. In the illustrated embodiment, the method 300 also includes a task 325 of splitting the data into test data 325(1), validation data 325(2), and training data 325(3). The method also includes a task 330 of identifying and customizing an artificial neural network (ANN) model and a task 335 of utilizing the training data 325(3) to train the ANN model. In the illustrated embodiment, the method 300 also includes a task 340 of optimizing the ANN model and a task 345 of validating the ANN model. Once the ANN model has been trained, optimized, and validated, the method 300 includes a task 350 of evaluating the test data 325(1). After the test data 325(1) has been evaluated utilizing the ANN, the method 300 includes a task 355 of integrating the data with wearables, a task 360 of deploying the model, and then a task 365 of outputting the real-world data. In this manner, the method 300 is configured to utilize artificial intelligence to transform the raw PPG data signal into clean data segments.

While this invention has been described in detail with particular references to exemplary embodiments thereof, the exemplary embodiments described herein are not intended to be exhaustive or to limit the scope of the invention to the exact forms disclosed. Persons skilled in the art and technology to which this invention pertains will appreciate that alterations and changes in the described structures and methods of assembly and operation can be practiced without meaningfully departing from the principles, spirit, and scope of this invention, as set forth in the following claims.

What is claimed is:

1. A biometric measurement device comprising:
a photodiode array comprising a plurality of photodiodes and at least one polarization filter covering at least one of the plurality of photodiodes; and
a light-emitting diode array comprising a plurality of light-emitting diodes proximate to the photodiode array;
a non-volatile memory device; and
a processor in electronic communication with the non-volatile memory device,
wherein the photodiode array is configured to receive a reflected waveform from light emitted from the light-emitting diode array, and
wherein the processor is configured to determine at least one physiological parameter based on a pulsatile (AC) component and a static (DC) component of the reflected waveform.

2. The biometric measurement device of claim 1, wherein the processor is further configured to determine one or more health metrics utilizing the at least one physiological parameter, wherein the one or more health metrics are selected from the group consisting of respiratory rate, heart rate, blood oxygen saturation, blood pressure, heart rate variability, maximal oxygen consumption, systolic volume, cardiac output, cardiac index, metabolic rate, and metabolic fitness.

3. The biometric measurement device of claim 1, wherein at least one of the plurality of light-emitting diodes is configured to emit polarized light.

4. The biometric measurement device of claim 1, further comprising at least one polarization filter on at least one of the plurality of light-emitting diodes.

5. The biometric measurement device of claim 1, wherein a first-emitting diode of the plurality of light-emitting diodes is configured to emit light having a first wavelength, and wherein a second-emitting diode of the plurality of light-emitting diodes is configured to emit light having a second wavelength different than the first wavelength.

6. The biometric measurement device of claim 1, wherein the plurality of light-emitting diodes is arranged in an annular configuration around the photodiode array.

7. The biometric measurement device of claim 1, wherein the plurality of photodiodes is arranged in an annular configuration around the light-emitting diode array.

8. The biometric measurement device of claim 1, wherein the plurality of photodiodes and the plurality of light-emitting diodes are arranged in a linear array.

9. The biometric measurement device of claim 1, wherein the non-volatile memory device comprises instructions which, when executed by the processor, cause the processor to:
activate a first light-emitting diode of the plurality of light-emitting diodes at a first time to emit light having a first wavelength;
receive, with at least one of the plurality of photodiodes, a first reflected waveform from the first light-emitting diode;
activate a second light-emitting diode of the plurality of light-emitting diodes at a second time to emit light having a second wavelength; and
receive, with at least one of the plurality of photodiodes, a second reflected waveform from the second light-emitting diode.

10. The biometric measurement device of claim 9, wherein the non-volatile memory device comprises instructions which, when executed by the processor, cause the processor to determine oxygen saturation SpO₂ as follows:
$${SpO}_{2} = α = β \cdot R$$
wherein α and β are constants, and wherein R is calculated as follows:
$$R = ( {AC}_{rms , λ1} / {DC}_{rms , λ1} ) / ( {AC}_{rms , λ2} / {DC}_{rms , λ2} )$$
where AC_{rms,λ1} is a root-mean-square of a pulsatile (AC) component of the first reflected waveform through one or more polarization filters collected from the light having the first wavelength λ₁; DC_{λ1} is a static (DC) component of the first reflected waveform through one or more polarization filters collected from the light having the first wavelength λ₁; AC_{rms,λ2} is a root-mean-square of a pulsatile (AC) component of the second reflected waveform collected through one or more polarization filters from the light having the second wavelength λ₂; and DC_{λ2} is a static (DC) component of the second reflected waveform through one of more polarization filters collected from the light having the second wavelength λ₂.

11. The biometric measurement device of claim 1, wherein the non-volatile memory device comprises instructions which, when executed by the processor, cause the processor to calculate a physiological parameter Sₜ at a time t as follows:
$${S}_{t} = f \frac{AC ( {λ}_{n , P} )}{DC ( {λ}_{n , P} )}$$
wherein ƒ is a function; AC(λ_{n,P}) is a pulsatile (AC) component of the reflected waveform collected with light from a light-emitting diode n having a wavelength λₙ and a polarization state P; DC(λ_{n,P}) is the static (DC) component of the reflected waveform collected with light from a light-emitting diode n having a wavelength λₙ, and the polarization state P.

12. The biometric measurement device of claim 9, wherein the polarization state P is selected from the group consisting of 0°, 30°, 45°, 60°, 90°, 120°, 135°, 150°, R, and L.

13. A method of performing photoplethysmography, the method comprising:
emitting light from at least one light-emitting diode of a plurality of light-emitting diodes arranged in a light-emitting diode array;
receiving a polarized waveform, with at least one photodiode of a plurality of photodiodes arranged in a photodiode array, from reflected light of the light emitted by the at least one light-emitting diode; and
determining at least one physiological parameter based on a pulsatile (AC) component and a static (DC) component of the polarized waveform.

14. The method of claim 13, wherein the emitting the light from the at least one light-emitting diode comprises emitting polarized light.

15. The method of claim 13, wherein the emitting the light from the at least one light-emitting diode comprises:
emitting a first light signal having a first wavelength from a first light-emitting diode of the plurality of light-emitting diodes at a first time;
receiving a first polarized waveform from a first reflected signal of the first light signal;
emitting a second light signal having a second wavelength from a second light-emitting diode of the plurality of light-emitting diodes at a second time different than the first time; and
receiving a second polarized waveform from a second reflected signal of the second light signal.

16. The method of claim 15, further comprising determining oxygen saturation SpO₂ as follows:
$${SpO}_{2} = α = β \cdot R$$
wherein α and β are constants, and wherein R is calculated as follows:
$$R = ( {AC}_{rms , λ1} / {DC}_{rms , λ1} ) / ( {AC}_{rms , λ2} / {DC}_{rms , λ2} )$$
where AC_{rms,λ1} is a root-mean-square of a pulsatile (AC) component of the first polarized waveform collected from the light having the first wavelength λ₁; DC_{λ1} is a static (DC) component of the first polarized waveform collected from the light having the first wavelength λ₁; AC_{rms,λ2} is a root-mean-square of a pulsatile (AC) component of the second polarized waveform collected from the light having the second wavelength λ₂; and DC_{λ2} is a static (DC) component of the second polarized waveform collected from the light having the second wavelength λ₂.

17. The method of claim 13, wherein the determining the at least one physiological parameter comprises determining signal Sₜ at a time t as follows:
$${S}_{t} = f \frac{AC ( {λ}_{n , P} )}{DC ( {λ}_{n , P} )}$$
wherein ƒ is a function; AC(λ_{n,P}) is a pulsatile (AC) component of the polarized waveform collected with light from a light-emitting diode n having a wavelength λₙ and a polarization state P; DC(λ_{n,P}) is the static (DC) component of the polarized waveform collected with light from a light-emitting diode n having a wavelength λₙ and the polarization state P.

18. The method of claim 17, wherein the polarization state P is selected from the group consisting of 0°, 30°, 45°, 60°, 90°, 120°, 135°, 150°, R, and L.

19. The method of claim 13, further comprising processing the polarized waveform with an artificial neural network model to improve accuracy and a wider range of healthcare metric prediction.
